(19) United States
(12) Patent Application Publication
(10) Pub. No.: US 2025/0090106 A1
(43) Pub. Date: Mar. 20, 2025

(54) PERSONALIZED HEMODYNAMIC TARGETS TO REDUCE HYPOTENSION DURING SURGERY

(71) Applicant: University of Pittsburgh - Of the Commonwealth System of Higher Education, Pittsburgh, PA (US)

(72) Inventors: Michael Schnetz, Newbury, OH (US); Aman Mahajan, Pittsburgh, PA (US); David Danks, La Jolla, CA (US)

(73) Assignee: University of Pittsburgh - Of the Commonwealth System of Higher Education, Pittsburgh, PA (US)

(21) Appl. No.: 18/294,504

(22) PCT Filed: Aug. 1, 2022
(86) PCT No.: PCT/US2022/039048

§ 371 Date: Feb. 1, 2024

Related U.S. Application Data
(60) Provisional application No. 63/228,528, filed on Aug. 2, 2021.

(51) Int. Cl. A61B 5/00; A61B 5/021

(52) U.S. Cl. CPC A61B 5/746; A61B 5/021; A61B 2505/05

(57) ABSTRACT

Methods are disclosed for generating a target mean arterial pressure (MAP) for a patient during a surgery. In some implementations, the disclosed methods include calculating the target MAP based on the age, biological sex, emergent surgery status, and American Society of Anesthesiology (ASA) physical status (PS) class of the patient, and a predetermined intraoperative hypotension (IOH) risk benchmark. Treating the patient to maintain a MAP at or above the target MAP during the surgery reduces a risk of IOH during the surgery, a risk of administering an unnecessary therapy to avoid IOH during the surgery; and a risk of not administering a therapeutic MAP-decreasing action during the surgery. In some implementations, the method further comprises conducting the surgery on the patient and treating the patient to maintain the MAP of the patient at or above the target MAP during the surgery. Also provided are systems for implementing the disclosed methods.

### CROSS REFERENCE TO RELATED APPLICATIONS

This application claims priority to U.S. Provisional Application No. 63/228,528, filed Aug. 2, 2021, which is incorporated by reference in its entirety.

### ACKNOWLEDGMENT OF GOVERNMENT SUPPORT

This invention was made with government support under grant numbers HL136836 and DA049630 awarded by the National Institutes of Health. The government has certain rights in the invention.

### BACKGROUND

Low blood pressure during surgery, known as intraoperative hypotension (IOH), increases the cost of perioperative care by billions of dollars each year in the United States. Approximately 100 million surgeries are performed annually and more than 70% are negatively impacted by IOH leading to severe organ system injuries including those affecting the heart, kidneys, and brain. IOH occurs because effective preventive strategies do not exist in the current standard of care. New methods that empower clinicians to safely and effectively reduce IOH exposure would make it possible to improve the quality of care at a lower cost by avoiding devastating complications and whose impact would reach each surgical patient of the future as well as the many health systems and hospitals delivering their care.

### SUMMARY

Provided herein are implementations of a novel method to generate a target MAP for initiating a MAP-increasing action for a patient during a surgery, as well as systems for implementing the method. Implementations of the disclosed method represent a shift from traditional hemodynamic management where IOH exposure (and its ill-effects) are not prevented, only limited by prompt recognition and treatment, to a new strategy where clinicians actively pursue personalized hemodynamic goals to prevent IOH before it occurs. This reduces the risk of IOH during surgery while at the same time reducing a risk of administering an unnecessary therapy to avoid IOH during the surgery, and reducing a risk of not administering a therapeutic MAP-decreasing action during the surgery.

In some implementations, a method of generating a target MAP for a patient during a surgery is provided. The method comprises calculating the target MAP based on the age, biological sex, emergent surgery status, and American Society of Anesthesiology (ASA) physical status (PS) class of the patient, and a predetermined IOH risk benchmark for the surgery. In some implementations, the surgery is a non-cardiac surgery under general anesthesia and the target MAP is a MAP falling within the range of from 66 to 100 mmHg (such as a MAP of 69 to 90 mmHg). Treating the patient to maintain a MAP at or above the target MAP during the surgery reduces a risk of IOH during the surgery (such as an IOH of 65 mmHg or less), a risk of administering an unnecessary therapy to avoid IOH during the surgery, and a risk of not administering a therapeutic MAP-decreasing action during the surgery.

In several implementations, the surgery comprises MAP-modulating treatment that increases or decreases MAP in the patient, for example, by increasing a MAP-increasing therapy, increasing MAP-decreasing therapy, decreasing MAP-increasing therapy, and/or decreasing MAP-decreasing therapy.

In several implementations, treating the patient to maintain the MAP of the patient at or above the target MAP during the surgery comprises administering or increasing a MAP-increasing therapy and/or reducing a MAP-decreasing therapy in an amount effective to maintain the MAP of the patient at or above the target MAP.

In some implementations, the method further comprises conducting the surgery on the patient and treating the patient to maintain the MAP of the patient at or above the target MAP during the surgery. In several such implementations, maintaining the MAP of the patient at or above the target MAP reduces the IOH risk for the patient compared to an average IOH risk for historical patients (such as prior patients from the same hospital system or surgical center as the patient) with comparable surgery and age, biological sex, emergent surgery status, and ASA PS class as the patient.

In some implementations, treating the patient to maintain the MAP of the patient at or above the target MAP reduces the risk of the IOH during the surgery, the risk of administering an unnecessary therapy to avoid IOH during the surgery, the risk of not administering a therapeutic MAP-decreasing action during the surgery, and/or the risk of surgical complication, compared to a suitable control. For example, the reduction in risk can be at least 50% compared to the control. The control can be, for example, an average IOH risk, an average risk of administering an unnecessary therapy to the patient to avoid IOH, an average risk of not administering a therapeutic MAP-decreasing action, and/or an average risk of surgical complication, for historical patients (such as prior patients from the same hospital system or surgical center as the patient) with comparable surgery and age, biological sex, emergent surgery status, and ASA PS class as the patient.

In some implementations, the method comprises generating a pre-, intra-, and/or post-surgical report comprising the target MAP and/or a risk curve showing different target MAPs for the patient based on different predetermined IOH risk benchmarks. In some implementations, the report is outputting to a user.

In some implementations, data representing the report or the target MAP and/or a risk curve showing different target MAPs for the patient based on different predetermined IOH risk benchmarks is stored in a computer-readable storage medium. In several implementations, the method comprises receiving data representing the calculated target MAP and/or a risk curve showing different target MAPs for the patient based on different predetermined IOH risk benchmarks prior to the surgical procedure for the current patient via a computer network.

Also provided is a system for monitoring a patient during a surgery, comprising a hardware unit including a hardware processor and system memory, a target MAP for the patient during the surgery stored in the system memory and calculated according to the methods provided herein, a hemodynamic sensor coupled to the hardware unit, and a sensory alarm. The hardware processor of the system is configured to activate the sensory alarm if a MAP measurement below the target MAP is measured for the patient during the surgery.

In additional implementations, computer systems, and computer readable media are provided that are configured to execute instructions to perform a method as described herein.

The foregoing and other features and advantages of the invention will become more apparent from the following detailed description of several implementations which proceeds with reference to the accompanying figures.

### BRIEF DESCRIPTION OF THE DRAWINGS

FIG. 1. Validation of exponential with theta model for MAP associated IOH risk in selected subgroups. The proportion of MAP value measurements followed by IOH within five minutes generated from the training dataset (dark grey dots) is compared to that of the validation dataset (light grey dots) with model superimposed (line). MAP mean arterial pressure. IOH, intraoperative hypotension. Subgroup definition: Column 1 (Emergent surgery yes-1, no-0), Column 2 (ASA PS class I-IV), Column 3 (Biological sex: Male/Female), Column 4 (Age Quartile Q1-4).

FIGS. 2A and 2B. Low risk MAPs identified using 5% risk benchmark in 0_1_Male_Q1 (FIG. 2A) and 0_3_Male_Q4 (FIG. 2B) subgroups. MAP-IOH risk model for both subgroups shown (line) where IOH risk within five minutes of MAP value measurement (y-axis) is plotted against MAP value (x-axis). High and low risk MAPs are distinguished by MAP target (vertical line), which is the lowest MAP in each model whose IOH risk is less than 5%. FIG. 2A. Low-risk MAPs are 73-100 mmHg in male patients assigned ASA PS I classification between the age of 18 and 42 years old undergoing non-emergent surgery. FIG. 2B. Low-risk MAPs are 82-100 mmHg in male patients older than 66 years of age assigned ASA PS III classification undergoing non-emergent surgery. MAP of 80 mmHg is low risk in patients represented in FIG. 2A (dot) but high risk in those represented by FIG. 2B (dot).

FIG. 3. Comparing MAP-IOH risk models using five and ten minute interval following MAP measurement to calculate IOH risk.

FIG. 4. Comparing MAP-IOH risk models between MAPs with different blood pressure trends preceding their measurement. Decreasing, increasing, and no change in MAP trends are shown.

FIG. 5. Comparing MAP-IOH risk models between MAPs with different prior IOH exposures defined by the number of MAPs <65 mmHg that occurred prior to their measurement. Exposure quartiles were defined as 0 events, 1-2 events, 3-5 events, and >5 events.

FIGS. 6A-6D. MAP-IOH risk models for select subgroups that vary by single characteristics. FIG. 6A—Emergent and non-emergent surgery in patients that were female, ASA physical status III, and greater than 66 years old. FIG. 6B—ASA physical status I-IV in patients that were male, between the age of 18-42 year old, and undergoing non-emergent surgery. FIG. 6C—Male and female biological sex in patients that were ASA physical status III, between the age of 57-66 years old, and undergoing non-emergent surgery. FIG. 6D—Age quartiles in patients that were female, ASA physical status III, and undergoing non-emergent surgery.

FIG. 7. illustrates a generalized example of a suitable computing environment in which described implementations, techniques, and technologies, including performing classification, can be exercised.

FIG. 8. is a diagram schematically depicting computing devices operating in conjunction with a computing cloud for implementation of disclosed technologies.

### DETAILED DESCRIPTION

### I. Introduction

Provided herein are implementations of a novel method to identify a specific therapeutic target MAP for individual patients predicted to reduced IOH exposure during surgery, as well as systems for implementing the method. The disclosed method, generated and validated using more than 100,000 surgeries, creates a target MAP for a patient before surgery from standard data inputs collected from patients and available in the electronic health record. The target MAP directs clinicians to maintain particular hemodynamic parameters during surgery that are within the clinically acceptable range defined by perioperative guidelines, but account for individual patient and procedure factors contributing to IOH risk. This technology represents a shift from traditional hemodynamic management where IOH exposure (and its ill-effects) are not prevented, only limited by prompt recognition and treatment, to a new strategy where clinicians actively pursue personalized hemodynamic goals to prevent IOH before it occurs. This reduces the risk of IOH during surgery while at the same time reducing the risk of administering an unnecessary therapy to avoid IOH during the surgery, and reducing the risk of not administering a therapeutic MAP-decreasing action during the surgery.

### II. Terms

Unless otherwise noted, technical terms are used according to conventional usage. In case of conflict, the present specification, including explanations of terms, will control. To facilitate review of the various implementations, the following explanations of terms are provided:

About: Plus or minus 5% from a reference amount. For example, “about 100” refers to 95 to 105.

Inhalation anesthetic: A drug administered via an inhalation route to an individual leading to reduced sensation of pain. Inhalation anesthetics are typically administered prior to and during surgical procedures to reduce or mask the pain of the surgical procedure. In some implementations, the inhalation anesthetic is a general anesthetic that induces a loss of consciousness. Non-limiting examples of inhalation anesthetics include isoflurane, desflurane, sevoflurane, and nitrous oxide.

Intraoperative hypotension (IOH): An occurrence of a MAP measurement below 65 mmHg in a patient during surgery.

Mean arterial pressure (MAP): The average arterial pressure during a single cardiac cycle in an individual. A target MAP for a patient during a surgical procedure is a MAP of 66 mmHg or above and whose risk of IOH within 10 minutes of its measurement is less than a clinically defined risk benchmark (such as a risk of 5% based on historical risk for a particular patient population).

Patient: Living multi-cellular vertebrate organisms, a category that includes both human and non-human mammals. In some examples, a patient is human surgical patient.

Surgical complication: A negative clinical outcome for a patient arising during or within a pre-determined period following a surgical procedure. The pre-determined time period is not more than one year and can begin immediately following surgery. In some examples, the pre-determined time period can be within one day, one week, one month, six months, or one year following surgery. Non-limiting examples of surgical complications include development of an infection, development or severity of pain (particularly post-surgical pain), time to achieve adequate level of post-surgical pain, development or severity of post-surgical nausea and/or vomiting, development or severity of post-surgical delirium, death within a selected time period following surgery (such as within one day, within one week, within one month or within one year), acute organ injury (such as acute kidney injury), respiratory failure, intraoperative awareness, acute anemia, thrombocytopenia, heart failure, coagulopathy, acidosis, malnutrition, sepsis, shock, and post-surgical hospital stay length of greater than average time.

Target MAP: An individualized MAP measure to maintain at or above for a patient during a surgical procedure. As described herein, maintenance of the MAP of the patient at or above the target MAP during the surgical procedure reduces the risk of IOH during the surgery, the risk of surgical complication during and following the surgery, and the risk of unnecessarily administering a MAP modulating therapy or not administering a beneficial MAP reducing therapy (such as increasing the dose of inhalation anesthetic) to the patient. A MAP reading below the target signals to the treating physician to initiate a MAP increasing therapy intended to bring the MAP of the patient back to a level at or above the target MAP.

Therapy: An intervention to a patient to achieve an intended clinical outcome for the benefit of the patient. For example, a MAP-increasing therapy is a clinical intervention to a patient to achieve an increase in patient MAP, such as administration of a therapeutical agent that increases blood-pressure, when there is a clinical need for an increase in MAP in the patient.

### III. Methods

Provided herein are implementations of a novel method for preoperative identification of a specific target MAP for individual patients that is predicted to reduced IOH exposure for the patient during surgery, as well as systems for implementing the method.

In some implementations, the method comprises preoperative generation of a target MAP for a patient during a surgery, comprising calculating the target MAP based on the age, biological sex, emergent surgery status, and ASA PS class of the patient, and a predetermined IOH risk benchmark for the patient during the surgery. In some implementations, the surgery is a non-cardiac surgery under general anesthesia.

In several implementations, the surgery comprises MAP-modulating action comprising a therapy that increases or decreases MAP in the patient. The target MAP is a MAP falling within the range of from 66 to 100 mmHg (such as 69 to 90 mmHg). Treating the patient to maintain a MAP at or above the target MAP during the surgery reduces the risk of IOH during the surgery (such as a MAP of 65 mmHg or less during the surgery, or a systolic blood pressure of 100 mmHg or less during surgery, and/or a decrease in blood pressure of at least 20% during surgery compared to preoperative blood pressure in the patient), the risk of administering an unnecessary therapy to the patient to avoid IOH during the surgery; and risk of not administering a therapeutic MAP-decreasing action to the patient during the surgery.

The predetermined intraoperative hypotension (IOH) risk benchmark may be determined according to any suitable standard, such as based on IOH risk (e.g., 1%, 5%, 10%; 15%, or from 1% to 15%, from 1% to 10%; or 10% or less, or 5% or less) for historical patients (such as prior patients from the same hospital system or surgical center) with comparable surgery and age, biological sex, emergent surgery status, and ASA PS class as the patient for the current surgery. In some implementations, the IOH risk percentage is calculated as the proportion of MAP readings between 65-100 mmHg followed by at least 1 MAP <65 mmHg within 10 minutes (such as within 5 minutes) of their measurement within patient-matched cohort, such as historical patients (such as prior patients from the same hospital system or surgical center) with comparable surgery and age, biological sex, emergent surgery status, and ASA PS class as the patient for the current surgery).

In some implementations, treating the patient to maintain the MAP of the patient at or above the target MAP during the surgery comprises administering or increasing a MAP-increasing therapy and/or reducing a MAP-decreasing therapy in an amount effective to maintain the MAP of the patient at or above the target MAP. For example, the MAP-increasing therapy can comprise administering fluids to increase blood pressure, administering a vasoactive agent (such as an inotrope and/or a vasopressor), or decreasing the administration of a vasodilator agent (such as nitroglycerin or a volatile anesthetic). For example, reducing the therapy that lowers MAP for the patient during the surgery can comprise reducing a dose of blood pressure-lowering agent (such as inhalation anesthetic) to the patient during the surgery. The method encompasses simultaneous administration of a MAP-increasing therapy and reduction of a MAP decreasing therapy to maintain the MAP of the patient at or above the target MAP during the surgery.

In some implementations, treating the patient to maintain the MAP of the patient at or above the target MAP reduces the risk of the IOH during the surgery compared to a control, for example a risk reduction of at least 50% (such as at least 60%, at least 70%, at least 80%, at least 90%, or at least 95%) compared to the control. In some such implementations, the control is an average IOH risk for historical patients (such as prior patients from the same hospital system or surgical center) with comparable surgery and age, biological sex, emergent surgery status, and ASA PS class as the patient.

In some implementations, treating the patient to maintain the MAP of the patient at or above the target MAP reduces the risk of administering an unnecessary therapy to the patient to avoid IOH during the surgery compared to a control, for example a risk reduction of at least 50% (such as at least 60%, at least 70%, at least 80%, at least 90%, or at least 95%) compared to the control. In some such implementations, the control is an average risk of administering an unnecessary therapy to avoid IOH during a surgery for historical patients (such as prior patients from the same hospital system or surgical center) with comparable surgery and age, biological sex, emergent surgery status, and ASA PS class as the patient.

In some implementations, treating the patient to maintain the MAP of the patient at or above the target MAP reduces the risk of not administering a therapeutic MAP-decreasing action during the surgery compared to a control, for example a risk reduction of at least 50% (such as at least 60%, at least 70%, at least 80%, at least 90%, or at least 95%) compared to the control. In some such implementations, the control is an average risk of not administering a therapeutic MAP-decreasing action during a surgery for historical patients (such as prior patients from the same hospital system or surgical center) with comparable surgery and age, biological sex, emergent surgery status, and ASA PS class as the patient.

In some implementations, treating the patient to maintain the MAP of the patient at or above the target MAP reduces the risk of surgical complication compared to a control, for example a risk reduction of at least 50% (such as at least 60%, at least 70%, at least 80%, at least 90%, or at least 95%) compared to the control. In some such implementations, the control is an average risk of a risk of surgical complication for historical patients (such as prior patients from the same hospital system or surgical center) with comparable surgery and age, biological sex, emergent surgery status, and ASA PS class as the patient. Non-limiting examples of such surgical complication include acute kidney injury, stroke, intraoperative awareness, cerebral vascular accident, transient ischemic attack, postoperative cognitive dysfunction, myocardial injury or infarction, need for interventional revascularization (such as percutaneous coronary intervention, or coronary artery bypass graft), hospitalization due to heart failure within 30 days or 1 year following surgery, or death within 30 days or 1 year following surgery.

In some implementations, the target MAP falls within a target MAP range that is calculated according to the patient status and target MAP ranges in Table 1.

TABLE 1
Exemplary target MAP ranges based on age, biological sex,
emergent surgery status, and ASA PS class.

| Patient Status | | | | |
|---|---|---|---|---|
| Emergent | ASA PS | Biological | | Target MAP |
| surgery? | Class | sex | Age | (mmHg) |
| No | 1 | F | 18-42 | 72-75 |
| No | 1 | M | 18-42 | 70-73 |
| No | 2 | F | 18-42 | 72-75 |
| No | 2 | F | 43-56 | 73-78 |
| No | 2 | F | 57-66 | 75-81 |
| No | 2 | F | >66 | 75-82 |
| No | 2 | M | 18-42 | 71-74 |
| No | 2 | M | 43-56 | 72-76 |
| No | 2 | M | 57-66 | 73-78 |
| No | 2 | M | >66 | 74-79 |
| No | 3 | F | 18-42 | 72-76 |
| No | 3 | F | 43-56 | 73-78 |
| No | 3 | F | 57-66 | 75-81 |
| No | 3 | F | >66 | 75-83 |
| No | 3 | M | 18-42 | 72-76 |
| No | 3 | M | 43-56 | 73-78 |
| No | 3 | M | 57-66 | 74-80 |
| No | 3 | M | >66 | 75-82 |
| No | 4 | F | 18-42 | 73-78 |
| No | 4 | F | 43-56 | 74-80 |
| No | 4 | F | 57-66 | 75-83 |
| No | 4 | F | >66 | 77-86 |
| No | 4 | M | 18-42 | 73-78 |
| No | 4 | M | 43-56 | 73-79 |
| No | 4 | M | 57-66 | 75-82 |
| No | 4 | M | >66 | 76-85 |
| Yes | 2 | M | 18-42 | 71-74 |
| Yes | 3 | F | >66 | 76-85 |
| Yes | 3 | M | 18-42 | 73-78 |
| Yes | 3 | M | 43-56 | 73-80 |
| Yes | 3 | M | 57-66 | 75-82 |
| Yes | 3 | M | >66 | 76-84 |
| Yes | 4 | M | 18-42 | 75-84 |
| Yes | 4 | M | 43-56 | 75-81 |
| Yes | 4 | M | 57-66 | 76-86 |
| Yes | 4 | M | >66 | 77-90 |

In some implementations, the target MAP is calculated according to the patient status and target MAP shown in Table 2.

TABLE 2
Target MAP ranges based on age, biological sex,
emergent surgery status, and ASA PS class.

| Patient Status | | | | |
|---|---|---|---|---|
| Emergent | ASA PS | Biological | | Target MAP |
| surgery? | Class | sex | Age | (mmHg) |
| No | 1 | F | 18-42 | 75 |
| No | 1 | M | 18-42 | 73 |
| No | 2 | F | 18-42 | 75 |
| No | 2 | F | 43-56 | 78 |
| No | 2 | F | 57-66 | 81 |
| No | 2 | F | >66 | 82 |
| No | 2 | M | 18-42 | 74 |
| No | 2 | M | 43-56 | 76 |
| No | 2 | M | 57-66 | 78 |
| No | 2 | M | >66 | 79 |
| No | 3 | F | 18-42 | 76 |
| No | 3 | F | 43-56 | 78 |
| No | 3 | F | 57-66 | 81 |
| No | 3 | F | >66 | 83 |
| No | 3 | M | 18-42 | 76 |
| No | 3 | M | 43-56 | 78 |
| No | 3 | M | 57-66 | 80 |
| No | 3 | M | >66 | 82 |
| No | 4 | F | 18-42 | 78 |
| No | 4 | F | 43-56 | 80 |
| No | 4 | F | 57-66 | 83 |
| No | 4 | F | >66 | 86 |
| No | 4 | M | 18-42 | 78 |
| No | 4 | M | 43-56 | 79 |
| No | 4 | M | 57-66 | 82 |
| No | 4 | M | >66 | 85 |
| Yes | 2 | M | 18-42 | 74 |
| Yes | 3 | F | >66 | 85 |
| Yes | 3 | M | 18-42 | 78 |
| Yes | 3 | M | 43-56 | 80 |
| Yes | 3 | M | 57-66 | 82 |
| Yes | 3 | M | >66 | 84 |
| Yes | 4 | M | 18-42 | 84 |
| Yes | 4 | M | 43-56 | 81 |
| Yes | 4 | M | 57-66 | 86 |
| Yes | 4 | M | >66 | 90 |

In some implementations, the method further comprises conducting the surgery on the patient and treating the patient to maintain the MAP of the patient at or above the target MAP during the surgery. The MAP of the patient is monitored during the surgery. If the MAP of the patient during the surgery is measured below the target MAP, then the patient is treated with a MAP-increasing action, such as administering or increasing a MAP-increasing therapy and/or reducing a MAP-decreasing therapy in an amount effective to maintain the MAP of the patient at or above the target MAP. In some implementations, reducing a MAP-decreasing therapy in an amount effective to maintain the MAP of the patient at or above the target MAP comprises reducing a dose of blood pressure-lowering agent to the patient during the surgery. If the MAP of the patient remains at or above the target MAP, then the MAP-increasing action is not initiated during the surgery (until or unless a MAP below the target MAP is measured). Further, if the MAP of the patient remains at or above the target MAP, then MAP-decreasing actions may be taken on the patient, such as an increase in anesthetic agent. Non-limiting examples of MAP-modulating agents that may be administered during the surgery include fluids, a vasoactive agent, a vasodilator agent, an anesthetic agent, an opioid, a hypnotic, and/or an ancillary agent with known cardiovascular effects (such as beta-blockers and anticholinergics) to the patient.

The surgical procedure comprises administration of an inhalation anesthetic to the patient. Non-limiting examples of such procedures include surgical procedures pertaining to ear-nose-throat surgery, trauma surgery, urological surgery, neurosurgery, orthopedic surgery, vascular surgery, thoracic surgery, pediatric surgery, cardiac surgery, OB-GYN surgery, ophthalmologic surgery, transplant surgery, general surgery, plastic surgery, colon and rectal surgery, gynecologic oncology surgery, oral and maxillofacial surgery, critical care procedures comprising inhalation anesthetics, and dental surgical services or dental surgical services.

Non-limiting examples of inhalation anesthetics for use in the surgical procedure include halothane, isoflurane, desflurane, sevoflurane, nitrous oxide, and combinations thereof. In some implementations, the inhalation anesthetic is administered to the patient as a general anesthetic.

In some implementations, a representation of the target MAP is outputted to a user or is stored in a computer readable storage medium. For example, the representation of the target MAP can be outputted to a user (such as a treating surgeon) in real-time, included in a pre- or post-surgical report, stored in one or more computer-readable storage media, or outputted to a secondary computing system or network.

### IV. Example Computing System and Environment

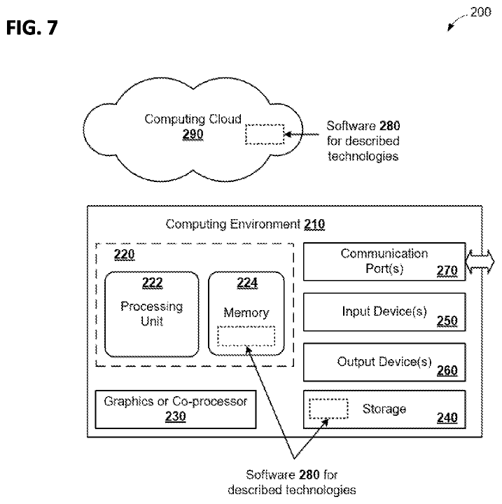

FIG. 7 illustrates a generalized example of a suitable computing system 200 for use in implementation of described examples, techniques, and technologies.

In some implementations, the system is implemented to generate a target MAP for initiating a MAP-increasing action for a patient during a surgery, comprising a step of calculating the target MAP based on the age, biological sex, emergent surgery status, and ASA PS class of the patient. For example, the computing system 200 can implement calculating the target MAP based on the age, biological sex, emergent surgery status, and ASA PS class of the patient based on appropriate reference, such as historical patients (such as prior patients from the same hospital system or surgical center as the patient) with comparable surgery and age, biological sex, emergent surgery status, and ASA PS class as the patient, or stored values, such as the target MAP range values or target MAP values provided in Tables 1 and 2.

In some implementations, the system is implemented to monitor a patient during a surgery. For example, the system comprises a hardware unit including a hardware processor and system memory, a target mean arterial pressure (MAP) for the patient during the surgery stored in the system memory and calculated according to the methods provided herein; a hemodynamic sensor coupled to the hardware unit; and a sensory alarm (for example, a visual alarm, an audible alarm, or a haptic alarm). The hemodynamic sensor is used to detect the MAP of the patient during the surgery, and the hardware processor is configured to activate the sensory alarm if a MAP below the target MAP is measured for the patient during the surgery. Activation of the sensory alarm alerts the treating physician of measurement of a MAP of the patient below the target MAP.

The hemodynamic sensor may be a non-invasive or minimally invasive sensor attached to the patient during the surgery. In one implementation, the hemodynamic sensor may be attached non-invasively at an extremity of the patient, such as a wrist, finger, arm, ankle, or toe of the patient. The hemodynamic sensor sends signals (e.g., wirelessly or via a wired connection) corresponding to the arterial pressure of the patient, from which a MAP value is calculated.

The computing system 200 is not intended to suggest any limitation as to scope of use or functionality of the technology, as the technology can be implemented in diverse general-purpose or special-purpose computing environments. For example, the disclosed technology can be implemented with other computer system configurations, including hand-held devices, multiprocessor systems, microprocessor-based or programmable consumer electronics, network PCs, minicomputers, mainframe computers, integrated anesthesia monitors, and the like. The disclosed technology can also be practiced in distributed computing environments where tasks are performed by remote processing devices that are linked through a communications network. In a distributed computing environment, program modules can be located in both local and remote memory storage devices.

With reference to FIG. 7, the computing environment 210 includes at least one central processing unit 222 and memory 224. In FIG. 7, this most basic configuration 220 is included within a dashed line. The central processing unit 222 executes computer-executable instructions for implementation for the methods and systems described herein and can be a real or a virtual processor. In a multi-processing system, multiple processing units execute computer-executable instructions to increase processing power and as such, multiple processors can be running simultaneously. The memory 224 can be volatile memory (e.g., registers, cache, RAM), non-volatile memory (e.g., ROM, EEPROM, flash memory, etc.), or some combination of the two. The memory 224 stores software 280 that can, for example, implement the technologies described herein. Computing environment 210 can also include a graphics processing unit or co-processing unit 230.

A computing environment can have additional features. For example, the computing environment 200 includes storage 240, one or more input devices 250, one or more output devices 260, and one or more communication connections 270. An interconnection mechanism (not shown) such as a bus, a controller, or a network, interconnects the components of the computing environment 200. Typically, operating system software (not shown) provides an operating environment for other software executing in the computing environment 200, and coordinates activities of the components of the computing environment 200.

The storage 240 can be removable or non-removable, and includes magnetic disks, magnetic tapes or cassettes, CD-ROMs, CD-RWs, DVDs, or any other medium which can be used to store information and that can be accessed within the computing environment 200. The storage 240 stores instructions for the software 280 and measurement data, which can implement technologies described herein.

The input device(s) 250 can be a touch input device, such as a keyboard, keypad, mouse, touch screen display, pen, or trackball, a voice input device, a scanning device, or another device, that provides input to the computing environment 210. The input device(s) 250 can also include interface hardware for connecting the computing environment to control and receive data from host and client computers, storage systems, measurement acquisition components, control excitation sources, or to display or output data processed according to methods disclosed herein, including data acquisition systems coupled to a plurality of sensors.

In some implementations, the computing system 200 includes one or more sensors for collecting the MAP measurements from the patient during the surgery. In some implementations, the computing system 200 includes one or more anesthetic data devices that generate data for the MAP measurements. In some implementations, the computing system 200 includes a display for real-time presentation of MAP measurement and/or target MAP for the patient during the surgery to a user. For example, the display can be configured for indicating to a treating anesthesiologist, anesthesia-care providers, and surgeon the status of a patient during a surgery. In some implementations, the computing system 200 includes one or more alarms that indicate when a MAP measurement for the patient is close to or has reached the target MAP for the patient during the surgery.

For audio, the input device(s) 250 can be a sound card or similar device that accepts audio input in analog or digital form, or a CD-ROM reader that provides audio samples to the computing environment 200. The output device(s) 260 can be a display, printer, speaker, CD-writer, or another device that provides output from the computing environment 210.

The communication connection(s) 270 enable communication over a communication medium (e.g., a connecting network) to another computing entity. The communication medium conveys information such as computer-executable instructions, compressed graphics information, video, or other data in a modulated data signal.

Some examples of the disclosed methods can be performed using computer-executable instructions implementing all or a portion of the disclosed technology in a computing cloud 290. For example, calculating the target MAP based on the age, biological sex, emergent surgery status, and ASA PS class of the patient as described herein can be executed locally in the computing environment, on remote servers located in the computing cloud 290, or the process can be partitioned and executed using both locations.

Computer-readable media are any available media that can be accessed within a computing environment 200. By way of example, and not limitation, with the computing environment 210, computer-readable media include memory 220 and/or storage 240. As should be readily understood, the term computer-readable storage media includes the media for data storage such as memory 220 and storage 240, and not transmission media such as modulated data signals.

The present innovations can be described in the general context of computer-executable instructions, such as those included in program modules, being executed in a computing system on a target real or virtual processor. Generally, program modules or components include routines, programs, libraries, software objects, classes, components, data structures, etc. that perform particular tasks or implement particular abstract data types. The functionality of the program modules can be combined or split between program modules as desired in various implementations. Computer-executable instructions for program modules can be executed within a local or distributed computing system.

In general, a computing system, computing environment, or computing device can be local or distributed, and can include any combination of special-purpose hardware and/or general-purpose hardware and/or virtualized hardware, together with software implementing the functionality described herein. Virtual processors, virtual hardware, and virtualized devices are ultimately embodied in one or another form of physical computer hardware.

### V. Example Cloud Computing Environment

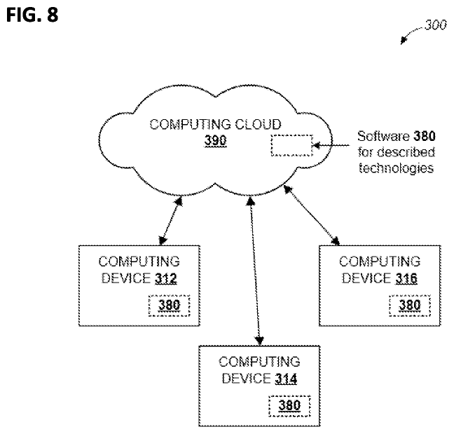

FIG. 8 depicts an example cloud computing environment 300 in which the described technologies can be implemented. The cloud computing environment 300 comprises a computing cloud 390 containing resources and providing services. The computing cloud 390 can comprise various types of cloud computing resources, such as computer servers, data storage repositories, networking resources, and so forth. The computing cloud 390 can be centrally located (e.g., provided by a data center of a business or organization) or distributed (e.g., provided by various computing resources located at different locations, such as different data centers and/or located in different cities or countries).

The computing cloud 390 can be operatively connected to various types of computing devices (e.g., client computing devices), such as computing devices 312, 314, and 316, and can provide a range of computing services thereto. One or more of computing devices 312, 314, and 316 can be computers (e.g., server, virtual machine, embedded systems, desktop, or laptop computers), mobile devices (e.g., tablet computers, smartphones, or wearable appliances), or other types of computing devices. Connections between computing cloud 390 and computing devices 312, 314, and 316 can be over wired, wireless, or optical links, or any combination thereof, and can be short-lived or long-lasting. These connections can be stationary or can move over time, being implemented over varying paths and having varying attachment points at each end. Computing devices 312, 314, and 316 can also be connected to each other.

Computing devices 312, 314, and 316 can utilize the computing cloud 390 to obtain computing services and perform computing operations (e.g., data processing, data storage, and the like). Particularly, software 380 for performing the described innovative technologies can be resident or executed in the computing cloud 390, in computing devices 312, 314, and 316, or in a distributed combination of cloud and computing devices.

### VI. General Considerations

As used in this application the singular forms “a,” “an,” and “the” include the plural forms unless the context clearly dictates otherwise. Additionally, the term “includes” means “comprises.” Furthermore, as used herein, the term “and/or” means any one item or combination of items in the phrase.

The systems, methods, and apparatus described herein should not be construed as being limiting in any way. Instead, this disclosure is directed toward all novel and non-obvious features and aspects of the various disclosed implementations, alone and in various combinations and subcombinations with one another. The disclosed systems, methods, and apparatus are not limited to any specific aspect or feature or combinations thereof, nor do the disclosed things and methods require that any one or more specific advantages be present or problems be solved. Furthermore, any features or aspects of the disclosed implementations can be used in various combinations and subcombinations with one another.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed things and methods can be used in conjunction with other things and methods.

Theories of operation, scientific principles, or other theoretical descriptions presented herein in reference to the apparatus or methods of this disclosure have been provided for the purposes of better understanding and are not intended to be limiting in scope. The apparatus and methods in the appended claims are not limited to those apparatus and methods that function in the manner described by such theories of operation.

Any of the disclosed methods can be implemented using computer-executable instructions stored on one or more computer-readable media (e.g., non-transitory computer-readable media, such as one or more optical media discs, volatile memory components (such as DRAM or SRAM), or nonvolatile memory components (such as flash drives or hard drives)) and executed on a computer (e.g., any commercially available computer, including smart phones or other mobile devices that include computing hardware). Any of the computer-executable instructions for implementing the disclosed techniques, as well as any data created and used during implementation of the disclosed implementations, can be stored on one or more computer-readable media (e.g., non-transitory computer-readable media). The computer-executable instructions can be part of, for example, a dedicated software application, or a software application that is accessed or downloaded via a web browser or other software application (such as a remote computing application). Such software can be executed, for example, on a single local computer (e.g., as a process executing on any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C, C++, Common Lisp, Dylan, Erlang, Fortran, Go, Haskell, Java, JavaScript, Julia, Python, Scheme, or any other suitable programming language. Likewise, the disclosed technology is not limited to any particular computer or type of hardware. Certain details of suitable computers and hardware are well-known and need not be set forth in detail in this disclosure.

Furthermore, any of the software-based implementations (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

Having described and illustrated the principles of the innovations in the detailed description and accompanying drawings, it will be recognized that the various examples can be modified in arrangement and detail without departing from such principles.

### VII. Examples

The following examples are provided to illustrate particular features of certain implementations, but the scope of the claims should not be limited to those features exemplified.

### Example 1

### Identification of Patient-Dependent, Intraoperative Blood Pressure Targets Associated with Differential Risks of Hypotension During Noncardiac Surgery

### Abstract

Background: Clinicians attempt to maintain intraoperative blood pressures above the thresholds used to define hypotension, however, limited evidence supports maintaining particular pressures. Here, a preoperative prediction model is developed to distinguish common intraoperative mean arterial pressures (MAP) according to their risk of hypotension and identify low risk pressures before surgery.

Background: Intraoperative hypotension (IOH) is responsible for organ system injuries and postoperative death. Blood pressure itself is a powerful predictor of IOH, however, it is unclear what range of pressures need to be targeted in different patient subgroups that prevent subsequent hypotension. The objective for this example was to develop a high-performance preoperative prediction model that classifies in patient subgroups mean arterial pressures (MAP) according to their risk of intraoperative hypotension and determines prior to surgery blood pressures that pose the least risk for IOH.

Methods: Adult, noncardiac surgeries at the University of Pittsburgh Medical Center were divided in training and validation cohorts, then assigned into smaller subgroups according to preoperative risk factors. Primary outcome was hypotension risk for individual MAP values 65 to 100 mmHg, defined as the proportion of a value's measurements followed within five or ten minutes by a MAP <65 mmHg. Risk was assessed in subgroups containing proportions with a CI<0.05 using five models. For the best fitting model, 1) performance was validated, 2) low risk MAP targets were identified according to applied benchmarks, and 3) preoperative risk factors were evaluated as predictors of model parameters.

Results: A total of 166,091 surgeries were included, with 121,032 and 45,059 surgeries containing 5.4 million and 1.9 million MAP measurements were included in the training and validation set, respectively. Thirty-six subgroups with at least twenty-one proportions (p<0.05) were identified, representing 92% and 94% of available MAP measurements, respectively. The exponential with theta constant model demonstrated the best fit (weighted sum of squared error 0.0005) and the mean squared error of hypotension risk per MAP did not exceed 0.01% in validation testing. MAP targets ranged between 69 and 90 mmHg depending on the subgroup and benchmark used. Increased age, higher American Society of Anesthesiologists physical status, and female biological sex independently predicted (p<0.05) hypotension risk curves with less rapid decay and higher plateaus.

Conclusions: Intraoperative hypotension risk specific to a particular MAP is patient-dependent, but predictable prior to surgery. The model can preoperatively identify MAPs that are less likely to produce IOH, potentially allowing clinicians to develop more personalized approaches for managing hemodynamics.

### Introduction

IOH in noncardiac surgery is a major risk factor for organ system injuries and postoperative death^{1,2}. MAP values of <65 mm Hg are typically used to define IOH and are strongly associated with poor post-operative outcomes³⁻⁸. Clinically, IOH is avoided by attempting to maintain pressures above these levels, however, a wide range of pressures are possible whose risks of adverse events or poor outcomes are poorly differentiated⁹, making it unclear if one pressure, or a particular range of pressures, should be maintained over others.

Growing evidence demonstrates blood pressure itself is a powerful predictor of IOH, suggesting individual pressures have distinct risks that impact a patient's hypotension exposure¹⁰⁻¹³. The Hypotension Prediction Index (HPI) is a novel algorithm that that uses the arterial pressure waveform to accurately predict IOH up to fifteen minutes before it occurs¹¹. In a gradient boosting machine-learning model predicting post-induction hypotension, the initial mean arterial pressure (MAP) was most critical to accurate prediction compared to other physiological features¹², while pre-induction systolic blood pressure is an independent risk factor for IOH¹³. Despite these advances, the IOH risk associated with individual blood pressures in a patient remains undefined. If defined, new blood pressure targets can be identified as those that 1) do not constitute IOH per se, and 2) possess a low risk of subsequent IOH occurrence. Targets would then represent blood pressure(s) with the lowest overall IOH risk for patients during surgery.

New predictive models that explicitly define blood pressure associated IOH risk are required to test this possibility. If designed to perform preoperatively, risk information can be assessed early, prior to surgery unlike HPI and similar models that require intraoperative data. Here, we generate a high-performance, preoperative MAP-IOH risk model by first comparing a pool of candidate models according to their fit across a wide-range of patient subgroups, each characterized by unique IOH risk factors. The best fitting model was selected for performance validation using a time-separated dataset. Finally, MAP targets, those associated with low IOH risk, were identified for each subgroup using absolute and historical risk benchmarks.

This example provides a new predictive model, designed to be performed preoperatively, that allows risk information to be assessed prior to surgery unlike HPI and similar models that are used intraoperatively. The high-performance preoperative MAP-IOH risk model was generated by first comparing a pool of candidate models according to their fit across a wide-range of patient subgroups, each characterized by unique IOH risk factors. The primary outcome was to define the IOH risk associated with individual MAP values between 65 and 100 mmHg. The best fitting model was selected for performance validation using a time-separated dataset. Finally, MAP targets associated with low IOH risk were identified for each patient subgroup using absolute and historical risk benchmarks.

### Methods

The R statistical software (R Foundation for Statistical Computing, version 3.5.3, Austria) was used for all described analyses¹⁵.

### Study Population

Patients age 18 years or older undergoing noncardiac surgery with general anesthesia at Presbyterian and Montefiore hospitals at the University of Pittsburgh Medical Center health system between June 2011 and August 2020 were selected for study.

### Training and Validation Datasets

Data from the following variables were extracted from the Cerner electronic health record system for each study surgical procedure: patient age, ASA PS, sex, emergent surgery, primary surgical procedure, and intraoperative MAP with measurement timestamp. MAPs <10 and >250 mmHg were considered artifacts and removed. MAPs in the dataset were derived from both invasive and noninvasive monitoring techniques. When a MAP measurement existed for both techniques at the same minute in time, the invasive measurement was used. MAPs are not recorded in our Cerner system more frequently than once per minute. The total dataset was divided into two sets prior to analysis. Data from June 2011-March 2018 were dedicated to model training and selection, data from April 2018-August 2020 were dedicated to performance validation. A larger fraction of the data was used for training to maximize the MAPs used to evaluate and compare models.

### Primary Outcome

The primary outcome was IOH risk associated with individual MAP values between 65 and 100 mmHg following their measurement during surgery. MAPs in this range represent common pressures above an IOH threshold frequently applied in noncardiac surgery (65 mmHg^{2,5,9}) that are difficult to otherwise differentiate in real-world practice. For a MAP value, IOH risk was defined as the proportion of its measurements followed by at least one MAP <65 mmHg within the five minutes. This time interval was selected because it distinguishes IOH risk following blood pressure measurement better than other (longer) intervals^{10,11}.

### Statistical Analysis

Descriptive statistics were generated for patient and procedure characteristics for both training and validation datasets. First, second (median), and third quartiles were calculated for continuous variables patient age and MAP. Proportions were calculated for categorical variables ASA physical status, emergency surgery, biological sex, and surgical specialty.

### Development of MAP-IOH Risk Model

We sought to generate a preoperative model predicting MAP-associated IOH risk. Age, ASA physical status, biological sex, and emergent surgery are independent predictors of IOH¹³ and were incorporated into the model. Each surgery in the training dataset was assigned a unique ‘subgroup ID’ defined by emergency surgery (1-yes, 0-no), ASA physical status (1, 2, 3, 4, or 5), biological sex (male/female), and age quartile. Age quartiles were defined as: Q1 (18-42), Q2 (43-56), Q3 (57-66), and Q4 (>66 years old). For each subgroup, the proportion of total MAP measurements followed by IOH was calculated for each MAP value 65 through 100 mmHg. Only subgroups with at least twenty-one proportions whose confidence interval was <0.05 were included in model fitting to eliminate the effect of low confidence estimates. This cut-off was selected as a balance between maximizing the number of MAP inputs available for model fitting, while limiting the number of subgroup IDs and total MAP data excluded. Since the true relationship between MAP and IOH risk is unknown, multiple models were generated and compared with the best fitting model selected for validation. Evaluated models are listed below and for each y=Proportion of MAP m measurements followed by IOH, and x=MAP m where m=single MAP value between 65 and 100 mmHg.

$$y = β x + alpha ( linear ) \qquad 1 )$$

$$logit ( y ) = β x + alpha ( logit ) \qquad 2 )$$

$$y = α e \hat{} ( β x ) ( exponential ) \qquad 3 )$$

$$y = α x ( β ) ( power ) \qquad 4 )$$

$$y = α e \hat{} ( β x ) + θ ( exponential with theta constant ) \qquad 5 )$$

Parameters estimates that minimized the sum of squared errors (SSE) for each subgroup were identified using nls( ) function in ‘stats’ package. The overall SSE for a model was determined by computing the weighted (by each subgroup's total number of MAP measurements) sum of SSEs for the best-fitting parameters for that model for each subgroup. Model fitness was given by this overall SSE; lower values represent better model fit.

Validation of MAP-IOH risk model

Performance of the best fitting model was tested using the validation dataset. MAP proportions were calculated and subgroups selected as described above for training dataset. For each validation subgroup, the SSE was calculated using the fitted curve generated for that subgroup in the training dataset. To compare SSEs between training and validation sets, a subgroup's SSE was divided by its total number of proportions to yield an average SE per MAP value.

Identification of MAP targets

MAP targets for each subgroup were identified in the training dataset using the best fitting model and according to absolute and historical risk benchmarks. Absolute benchmarks were defined as 5%, 10%, and 15% IOH risk and chosen to highlight subgroup differences. Historical benchmarks were defined for individual subgroups as the IOH risk calculated from all MAPs between 65 and 100 mmHg, representing the overall risk associated with this blood pressure range. MAP targets were defined as the lowest MAP not to exceed benchmark risks.

### Post-Hoc Analyses

### Evaluating MAP Associated IOH Risk According to Differences in Monitoring Frequency, Preceding Blood Pressure Trends, and Prior IOH Exposure

MAP is a standard monitor, but its measurement frequency varies between patients. MAPs followed by more subsequent MAPs are more likely to capture IOH compared to those followed by less. To evaluate this possibility, the number of MAPs recorded in the five-minute interval used to calculate IOH risk was compared between MAP values 65 through 100 mmHg in the training dataset. The first, second, and third quartiles were calculated and reported. In addition, the model's ability to capture IOH risk associated with multiple subsequent blood pressures was tested by comparing models generated with five and ten minute intervals.

Factors that occur intraoperatively or whose effect on IOH risk is unclear (e.g. surgical procedure) were not included in model development, yet may still influence MAP associated IOH risk. To compare MAP associated IOH risk in instances where the preceding blood pressure is changing, MAPs in the training dataset were assigned as 1) increasing, 2) decreasing, or 3) unchanged according to the MAP that most immediately preceded it within the prior ten minutes. For example, a MAP of 75 mmHg would be assigned as ‘increasing’ if the MAP before it was 72 mmHg, but ‘decreasing’ if that MAP's value was 78 mmHg. Models comparing assignment groups were generated.

Finally, prior IOH may predict future IOH and this may be reflected in the model where MAPs associated with the highest risk are also associated with the greatest prior IOH exposure compared to lower risk MAPs. To evaluate this possibility, MAPs in the training dataset were assigned into quartiles of exposure defined by the number of MAP <65 mmHg that occurred prior to their measurement relative to all measurements. Models comparing exposure groups were generated.

IOH risk factors as predictors of MAP-IOH risk curve segments Alpha, beta, and theta parameters modulate specific portions of the MAP-IOH risk curve as defined by the exponential with theta constant model. Alpha and theta define the y- and x-axis asymptotes, respectively, beta defines how rapidly IOH risk changes across MAPs (i.e. ‘sharpness’ of the curve's elbow). To evaluate patient age, biological sex, emergent surgery, and ASA physical status as predictors of distinct portions of the curve, factors were analyzed as independent predictors of each parameter using the lm( ) function in ‘stats’ package.

### Results

### Training and Validation Datasets

A small fraction of surgeries (<0.3%) lacked intraoperative MAP data within 65-100 mmHg, and these were excluded from analysis. The patient and procedure factors characterizing the training and validation datasets are shown in Table 3. Representing 121,032 surgeries, patients in the training set included majority men (53.9%) with a median age of 56 years old, assigned an ASA physical status III classification, and undergoing a general surgery procedure. Patients in the validation set, composed of 45,059 surgeries, exhibited the same characteristics, but with a median age of 58 years old. Emergent surgery represented 13.5 and 15.7% of all surgeries in each set, respectively. The training set contained 6,987,790 total MAP measurements with a median MAP of 80 mmHg; 77.9% were between 65 and 100 mmHg. The validation set contained 2,566,291 total MAP measurements with a median MAP of 81 mmHg; 77.7% were between 65 and 100 mmHg.

TABLE 3
Patient and Procedure Characteristics in Training and Validation Datasets.

| Clinical demographic | Training (n = 121,032) | Validation (n = 45,059) |
|---|---|---|
| Age, year, Median (Q1-Q3) | 56 (43-67) | 58 (43-68) |
| ASA physical status I (%) | 3.6 | 3.8 |
| II | 29.2 | 21.5 |
| III | 51.2 | 51.9 |
| IV | 15 | 21.7 |
| V | 0.8 | 0.9 |
| Emergent (%) | 13.5 | 15.7 |
| Male biological sex (%) | 53.9 | 56.7 |
| Surgical Specialty (%) | General (24.3) | General (21.5) |
| | Neurological (18.7) | Orthopedic (18.7) |
| | Orthopedic (18.0) | Neurological (17.2) |
| | Thoracic (9.8) | Thoracic (10.6) |
| | Otolarynology (9.2) | Otolaryngology (6.5) |
| MAP, mmHg, Median (Q1-Q3) | 80 (72-91) | 81 (72-92) |
| Total MAP measurements | 6,987,790 | 2,566,291 |
| Total MAPs between 65-100 mmHg | 5,449,316 (77.9) | 1,994,869 (77.7) |
| (% of total measurements) | | |

ASA—American Society of Anesthesiologists,
MAP—mean arterial pressure

### MAP-IOH Risk Model Development and Validation

Thirty-six subgroups with at least twenty-one MAP proportions whose confidence interval was <0.05 were identified in both datasets (Table 4). These were termed high-confidence proportions. Subgroups represented 76.5 and 88.5% of all high-confidence proportions and 92.2 and 94.0% of total MAP measurements between 65 and 100 mmHg in training and validation sets, respectively.

TABLE 4
Total number of high confidence MAP proportions (CI < 0.05) and MAP
measurements for subgroups used in model development and validation.
Totals are shown for both training (Train) and validation (Valid) datasets.

| | | MAP | | MAP |
|---|---|---|---|---|
| | High Confidence | measurements | High Confidence | measurements |
| | MAP Proportions | (65-100) Train, | MAP Proportions | (65-100) Valid, |
| ID | Train, Total Count | Total Count | Valid, Total Count | Total Count |
| 0_1_Female_Q1 | 36 | 54767 | 33 | 21164 |
| 0_1_Male_Q1 | 36 | 72502 | 34 | 39102 |
| 0_2_Female_Q1 | 36 | 277294 | 36 | 79631 |
| 0_2_Female_Q2 | 36 | 237838 | 35 | 57938 |
| 0_2_Female_Q3 | 36 | 152195 | 31 | 38853 |
| 0_2_Female_Q4 | 36 | 96073 | 29 | 27988 |
| 0_2_Male_Q1 | 36 | 313017 | 36 | 99129 |
| 0_2_Male_Q2 | 36 | 229423 | 34 | 55523 |
| 0_2_Male_Q3 | 36 | 147238 | 32 | 41819 |
| 0_2_Male_Q4 | 36 | 81050 | 30 | 27365 |
| 0_3_Female_Q1 | 36 | 180262 | 36 | 71087 |
| 0_3_Female_Q2 | 36 | 305312 | 36 | 106312 |
| 0_3_Female_Q3 | 36 | 315619 | 36 | 118497 |
| 0_3_Female_Q4 | 36 | 395955 | 36 | 142433 |
| 0_3_Male_Q1 | 36 | 194472 | 36 | 78453 |
| 0_3_Male_Q2 | 36 | 357044 | 36 | 118025 |
| 0_3_Male_Q3 | 36 | 390384 | 36 | 158719 |
| 0_3_Male_Q4 | 36 | 434264 | 36 | 176693 |
| 0_4_Male_Q2 | 36 | 63536 | 33 | 35725 |
| 0_4_Male_Q3 | 36 | 83024 | 36 | 52327 |
| 0_4_Male_Q4 | 36 | 97091 | 36 | 62711 |
| 0_4_Female_Q4 | 35 | 65662 | 32 | 36576 |
| 0_4_Female_Q3 | 34 | 48530 | 29 | 26539 |
| 0_4_Male_Q1 | 34 | 42424 | 29 | 20666 |
| 1_2_Male_Q1 | 34 | 48380 | 27 | 14254 |
| 0_4_Female_Q2 | 33 | 41415 | 29 | 20044 |
| 1_4_Male_Q3 | 33 | 34415 | 28 | 20603 |
| 0_4_Female_Q1 | 32 | 27280 | 27 | 15818 |
| 1_3_Male_Q1 | 32 | 30623 | 23 | 12547 |
| 1_3_Male_Q2 | 32 | 34019 | 24 | 13469 |
| 1_3_Male_Q3 | 31 | 30847 | 25 | 13694 |
| 1_4_Male_Q2 | 31 | 28631 | 24 | 15252 |
| 1_3_Male_Q4 | 30 | 28962 | 21 | 12334 |
| 1_4_Male_Q1 | 30 | 23440 | 23 | 13158 |
| 1_4_Male_Q4 | 30 | 32758 | 26 | 19758 |
| 1_3_Female_Q4 | 28 | 28157 | 21 | 10784 |
| | % Total | % Total MAP | % Total | % Total MAP |
| | HighConfidence | measurements | HighConfidence | measurements |
| | MAP Proportions, | (65-100), | MAP Proportions, | (65-100), |
| | Training | Training | Validation | Validation |
| | 76.5 | 92.2 | 88.5 | 94.0 |

CI—confidence interval,
MAP—Mean arterial pressure
Subgroup ID definitions: Column 1 (Emergent yes-1, no-0),
Column 2 (ASA PS class I-IV),
Column 3 (Biological sex Male/Female),
Column 4 (Age Quartile Q1-4)

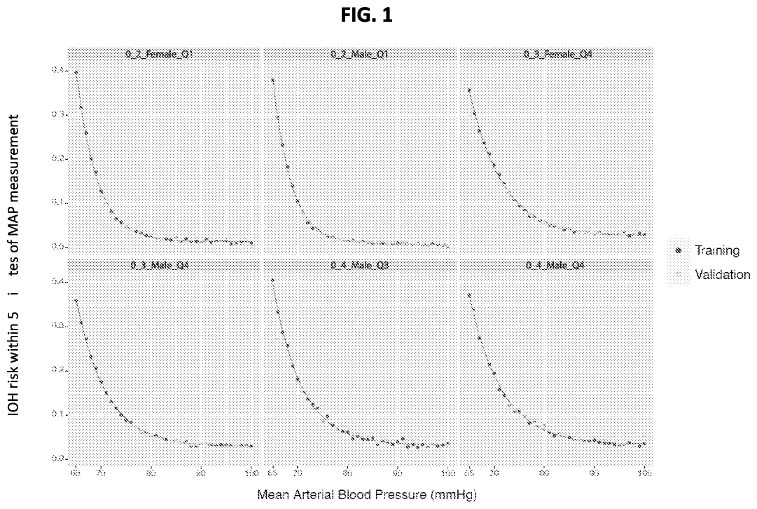

The sum of SSEs for each model is shown in Table 5. The linear model demonstrated the poorest fit with a weighted SSE of 0.0998. The exponential with theta constant model had the best fit with a weighted SSE of 0.0005. FIG. 1 shows performance validation of the exponential with theta constant model in select subgroups. IOH risk exponentially decreases as MAP increases from 65 mmHg before plateauing at higher MAPs. The rate of exponential decrease, the MAP at which risk plateaus, and the risk associated with individual MAP values dependents on model parameters (alpha, beta, theta) that define the fitted curve for each subgroup. In Table 6, the average squared error (SE) per MAP value is compared between datasets for each subgroup. The average SE per MAP value did not exceed 1.0×10E5 in the training set, which the model was fitted, and 1.0×10E4 in the validation set.

TABLE 5
Weighted SSE for 36 subgroups in each model

| Model | Weighted SSE |
|---|---|
| Linear | 0.09984 |
| Logit | 0.00803 |
| Exponential | 0.00550 |
| Power | 0.00386 |
| Exponential with theta constant | 0.00059 |

SSE—Sum of Squared Error

TABLE 6
Average standard error per MAP value in model training
and validation datasets for identified 36 subgroups.

| | Average SE per MAP | Average SE per MAP |
|---|---|---|
| ID | Proportion-Training | Proportion-Validation |
| 0_1_Female_Q1 | 2.70E−05 | 1.38E−04 |
| 0_1_Male_Q1 | 2.14E−05 | 3.15E−05 |
| 0_2_Female_Q1 | 1.14E−05 | 6.19E−05 |
| 0_2_Female_Q2 | 9.49E−06 | 2.03E−04 |
| 0_2_Female_Q3 | 3.40E−05 | 7.27E−05 |
| 0_2_Female_Q4 | 4.66E−05 | 1.09E−04 |
| 0_2_Male_Q1 | 1.06E−05 | 3.53E−05 |
| 0_2_Male_Q2 | 1.83E−05 | 7.18E−05 |
| 0_2_Male_Q3 | 1.49E−05 | 4.45E−05 |
| 0_2_Male_Q4 | 5.26E−05 | 9.37E−05 |
| 0_3_Female_Q1 | 1.03E−05 | 1.61E−04 |
| 0_3_Female_Q2 | 1.17E−05 | 9.60E−05 |
| 0_3_Female_Q3 | 1.44E−05 | 9.51E−05 |
| 0_3_Female_Q4 | 1.29E−05 | 1.54E−04 |
| 0_3_Male_Q1 | 1.11E−05 | 1.80E−04 |
| 0_3_Male_Q2 | 5.52E−06 | 8.59E−05 |
| 0_3_Male_Q3 | 8.45E−06 | 2.05E−04 |
| 0_3_Male_Q4 | 6.26E−06 | 2.23E−04 |
| 0_4_Female_Q1 | 3.18E−05 | 7.12E−05 |
| 0_4_Female_Q2 | 2.78E−05 | 1.38E−04 |
| 0_4_Female_Q3 | 3.85E−05 | 1.01E−04 |
| 0_4_Female_Q4 | 3.09E−05 | 6.33E−05 |
| 0_4_Male_Q1 | 4.95E−05 | 9.75E−05 |
| 0_4_Male_Q2 | 2.26E−05 | 4.95E−05 |
| 0_4_Male_Q3 | 2.85E−05 | 2.79E−04 |
| 0_4_Male_Q4 | 2.52E−05 | 7.35E−05 |
| 1_2_Male_Q1 | 1.89E−05 | 1.41E−04 |
| 1_3_Female_Q4 | 4.63E−05 | 1.08E−04 |
| 1_3_Male_Q1 | 4.95E−05 | 1.78E−04 |
| 1_3_Male_Q2 | 3.72E−05 | 9.56E−05 |
| 1_3_Male_Q3 | 4.76E−05 | 1.32E−04 |
| 1_3_Male_Q4 | 6.84E−05 | 7.53E−05 |
| 1_4_Male_Q1 | 7.38E−05 | 1.29E−04 |
| 1_4_Male_Q2 | 8.22E−05 | 1.23E−04 |
| 1_4_Male_Q3 | 5.99E−05 | 1.00E−04 |
| 1_4_Male_Q4 | 9.10E−05 | 8.64E−05 |

Subgroup ID definitions: Column 1 (Emergent yes-1, no-0),
Column 2 (ASA PS class I-IV),
Column 3 (Biological sex Male/Female),
Column 4 (Age Quartile Q1-4)

### MAP Targets

MAP targets, the minimum MAP with lower IOH risk than applied risk benchmarks, are shown in Table 7 for select subgroups; all targets are reported in Table 8. Targets vary from 69 to 90 mmHg depending on subgroup and benchmark. For example, a MAP of 73 mmHg is required to achieve less than 5% OH risk in 0_1_Male_Q1 patients, but 90 mmHg in 1_4_Male_Q4 patients. Historical risk benchmarks varied between 5.2% and 12.2% and targets based on these extend from 72 to 77 mmHg.

TABLE 7
MAP targets defined according to absolute
and historical risk benchmarks

| | IOH risk | IOH risk | IOH risk | | Historical |
|---|---|---|---|---|---|
| | (5%), | (10%), | (15%), | Historical | target, |
| ID | mmHg | mmHg | mmHg | IOH risk | mmHg |
| 0_1_Male_Q1 | 73 | 70 | 69 | 5.2% | 72 |
| 0_3_Male_Q4 | 82 | 75 | 72 | 8.6% | 76 |
| 0_3_Female_Q3 | 81 | 75 | 72 | 8.3% | 76 |
| 0_4_Female_Q4 | 86 | 77 | 73 | 10.5% | 76 |
| 1_4_Male_Q4 | 90 | 77 | 73 | 12.2% | 75 |

In Table 7, MAP target represents the minimum MAP whose IOH risk is less than risk benchmark. Absolute risk benchmarks were defined as 5, 10, and 15%. Historical risk benchmark is defined as the overall IOH risk of all MAPs between 65 and 100 mmHg for a subgroup in the training set. Subgroup ID definitions: Column 1 (Emergent surgery yes-1, no-0), Column 2 (ASA PS I-IV), Column 3 (Biological sex Male/Female), Column 4 (Age Quartile Q1-4). MAP—mean arterial pressure, IOH—intraoperative hypotension.

TABLE 8
MAP targets defined according to absolute and historical
risk benchmarks for all 36 subgroups.

| | IOH risk | IOH risk | IOH risk | | Historical |
|---|---|---|---|---|---|
| | (5%), | (10%), | (15%), | Historical | target, |
| ID | mmHg | mmHg | mmHg | IOH risk | mmHg |
| 0_1_Female_Q1 | 75 | 72 | 70 | 9.5% | 72 |
| 0_1_Male_Q1 | 73 | 70 | 69 | 5.2% | 72 |
| 0_2_Female_Q1 | 75 | 72 | 70 | 7.9% | 73 |
| 0_2_Female_Q2 | 78 | 73 | 71 | 8.2% | 74 |
| 0_2_Female_Q3 | 81 | 75 | 72 | 9.2% | 75 |
| 0_2_Female_Q4 | 82 | 75 | 72 | 8.9% | 76 |
| 0_2_Male_Q1 | 74 | 71 | 69 | 5.2% | 73 |
| 0_2_Male_Q2 | 76 | 72 | 70 | 6.3% | 74 |
| 0_2_Male_Q3 | 78 | 73 | 71 | 7.6% | 75 |
| 0_2_Male_Q4 | 79 | 74 | 71 | 8.0% | 75 |
| 0_3_Female_Q1 | 76 | 72 | 70 | 7.4% | 74 |
| 0_3_Female_Q2 | 78 | 73 | 71 | 7.5% | 75 |
| 0_3_Female_Q3 | 81 | 75 | 72 | 8.4% | 76 |
| 0_3_Female_Q4 | 83 | 75 | 72 | 8.4% | 77 |
| 0_3_Male_Q1 | 76 | 72 | 70 | 7.3% | 74 |
| 0_3_Male_Q2 | 78 | 73 | 71 | 7.4% | 75 |
| 0_3_Male_Q3 | 80 | 74 | 71 | 8.4% | 75 |
| 0_3_Male_Q4 | 82 | 75 | 72 | 8.7% | 76 |
| 0_4_Female_Q1 | 78 | 73 | 71 | 8.7% | 74 |
| 0_4_Female_Q2 | 80 | 74 | 71 | 9.2% | 75 |
| 0_4_Female_Q3 | 83 | 75 | 72 | 10.2% | 75 |
| 0_4_Female_Q4 | 86 | 77 | 73 | 10.6% | 76 |
| 0_4_Male_Q1 | 78 | 73 | 71 | 8.9% | 74 |
| 0_4_Male_Q2 | 79 | 73 | 71 | 9.3% | 74 |
| 0_4_Male_Q3 | 82 | 75 | 72 | 10.5% | 75 |
| 0_4_Male_Q4 | 85 | 76 | 72 | 10.6% | 75 |
| 1_2_Male_Q1 | 74 | 71 | 69 | 6.4% | 73 |
| 1_3_Female_Q4 | 85 | 76 | 72 | 10.0% | 76 |
| 1_3_Male_Q1 | 78 | 73 | 70 | 8.5% | 74 |
| 1_3_Male_Q2 | 80 | 73 | 71 | 8.7% | 75 |
| 1_3_Male_Q3 | 82 | 75 | 71 | 9.6% | 75 |
| 1_3_Male_Q4 | 84 | 76 | 73 | 10.0% | 76 |
| 1_4_Male_Q1 | 84 | 75 | 70 | 10.3% | 75 |
| 1_4_Male_Q2 | 81 | 75 | 72 | 10.4% | 74 |
| 1_4_Male_Q3 | 86 | 76 | 72 | 11.5% | 75 |
| 1_4_Male_Q4 | 90 | 77 | 73 | 12.2% | 75 |

In Table 8, MAP target represents the minimum MAP whose IOH risk is less than risk benchmark. Absolute risk benchmarks were defined as 5, 10, and 15%. Historical risk benchmark is defined as the overall IOH risk of all MAPs between 65 and 100 mmHg for a subgroup in the training set. Subgroup ID definitions: Column 1 (Emergent surgery yes-1, no-0), Column 2 (ASA PS I-IV), Column 3 (Biological sex Male/Female), Column 4 (Age Quartile Q1-4). MAP—mean arterial pressure, IOH—intraoperative hypotension.

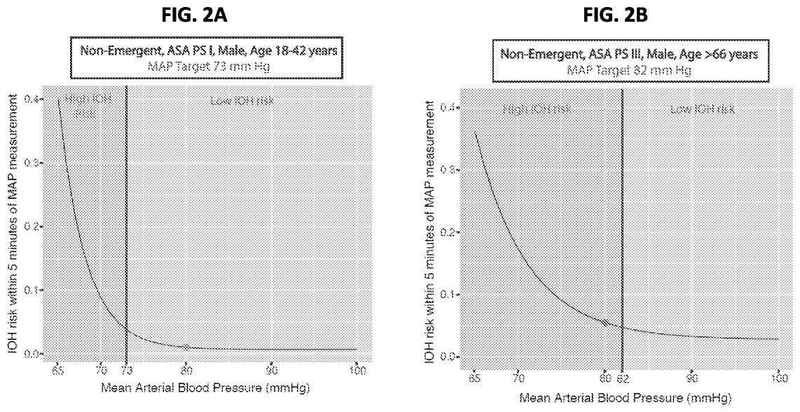

FIG. 2 compares low risk MAPs, identified with the 5% risk benchmark, between 0_1_Male_Q1 and 0_3_Male_Q4 patients. A wider range of MAPs are low risk in 0_1_Male_Q1 patients (73-100 mmHg) compared to 0_3_Male_Q4 patients (82-100 mmHg). A MAP of 80 mmHg, well above 65 mmHg IOH threshold, is considered low risk in the former subgroup (dot), but high risk in the latter (dot).

### Post-Hoc Analyses

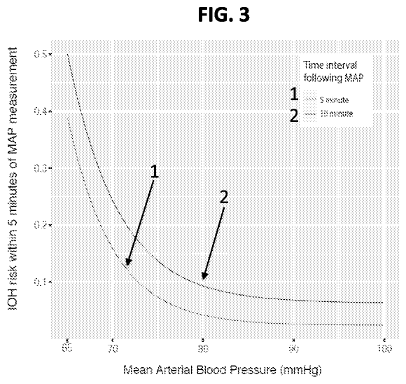

The interquartile range for the number of MAPs recorded within the five-minute interval following MAP measurement is uniform across all MAP values and shown in Table 9. Most MAPs were followed by two subsequent MAPs. All MAP values were associated with a higher IOH risk in a model generated with a ten-minute interval compared to the five-minute interval model (FIG. 3), demonstrating the model distinguishes IOH risk of multiple blood pressures following MAP measurement and beyond only those captured in a five minute period. The relative risk of a given MAP is similarly distinguished in each model.

TABLE 9
Distribution of the number of MAP measurements recorded
following each MAP value between 65 and 100 in training dataset.

| MAP (mmHg) | 1st Quartile | Median | 3rd Quartile |
|---|---|---|---|
| 65 | 1 | 2 | 2 |
| 66 | 1 | 2 | 2 |
| 67 | 1 | 2 | 2 |
| 68 | 1 | 2 | 2 |
| 69 | 1 | 2 | 2 |
| 70 | 1 | 2 | 2 |
| 71 | 1 | 2 | 2 |
| 72 | 1 | 2 | 2 |
| 73 | 1 | 2 | 2 |
| 74 | 1 | 2 | 2 |
| 75 | 1 | 2 | 2 |
| 76 | 1 | 2 | 2 |
| 77 | 1 | 2 | 2 |
| 78 | 1 | 2 | 2 |
| 79 | 1 | 2 | 2 |
| 80 | 1 | 2 | 2 |
| 81 | 1 | 2 | 2 |
| 82 | 1 | 2 | 2 |
| 83 | 1 | 1 | 2 |
| 84 | 1 | 1 | 2 |
| 85 | 1 | 1 | 2 |
| 86 | 1 | 1 | 2 |
| 87 | 1 | 1 | 2 |
| 88 | 1 | 1 | 2 |
| 89 | 1 | 1 | 2 |
| 90 | 1 | 1 | 2 |
| 91 | 1 | 1 | 2 |
| 92 | 1 | 1 | 2 |
| 93 | 1 | 2 | 2 |
| 94 | 1 | 2 | 2 |
| 95 | 1 | 2 | 2 |
| 96 | 1 | 2 | 2 |
| 97 | 1 | 2 | 2 |
| 98 | 1 | 2 | 2 |
| 99 | 1 | 2 | 2 |
| 100 | 1 | 2 | 2 |

MAP—Mean arterial pressure

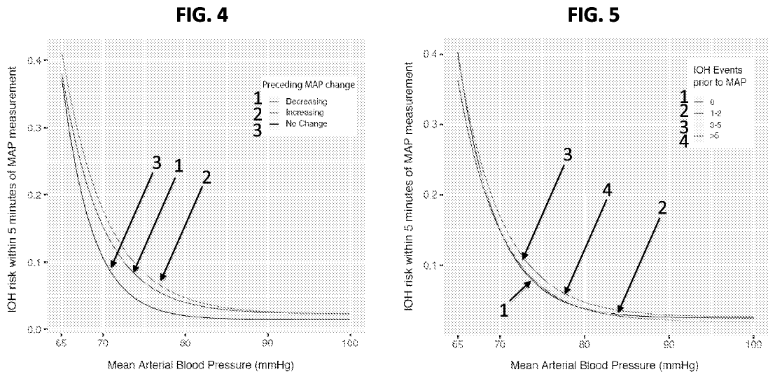

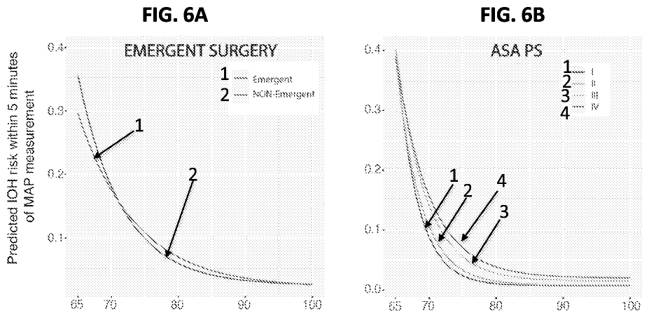

Models comparing MAP associated IOH risk between different preceding MAP trends and IOH exposures are shown in FIGS. 4 and 5, respectively. Preceding MAP increases and decreases are associated with higher IOH risks across all MAP values compared to no change. For MAPs 65 to approximately 85 mmHg, an increasing trending is associated with larger risk than decreasing or no change trends. The maximum difference at any given MAP is less than 10% and decreases at both ends of the MAP range. The MAP-IOH risk changes little between prior IOH exposure groups. MAPs with the highest prior exposure are associated with no more than 2% greater risk at any MAP value compared to MAPs with other exposures and whose risk curves are poorly distinguished from one another.

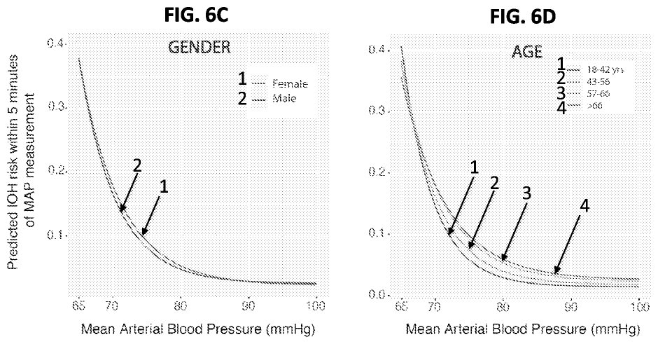

Age, biological sex, and ASA physical status are independent predictors of the beta model parameter, whereas only age and ASA physical status are independent predictors of the theta parameter (Table 10, FIGS. 6A-6D). Alpha and beta parameters are tightly correlated (>0.99), and alpha shared the same predictors as beta. These differences are exemplified in MAP-IOH risk curves between subgroups that vary by a single characteristic. The beta parameter defines how quickly risk increases from a plateau at higher MAPs, defined by theta, as MAP decreases towards 65 mmHg. Young age, male biological sex, and lower ASA physical status classification predict MAP-IOH risk curves with sharp ‘elbows’, meaning the plateau portion of the curve transitions to the exponential portion more rapidly and at a lower MAP compared to curves of other patients. IOH risk only begins to exponentially increase at MAPs closest to 65 mmHg in patients with these characteristics. Older age and higher ASA physical status predict risk curves with higher plateaus, meaning IOH risk at higher MAPs is greater in patients with these characteristics compared those younger and with lower physical status assignments.

TABLE 10
Linear regression analysis of Age, Biological sex, ASA Physical
Status, and Emergent surgery on Beta and Theta parameters.

| | | Coefficient | | | |
|---|---|---|---|---|---|
| Parameter | Predictor | Estimate | SE | p-value | p < 0.05 |
| BETA | Emergent | 0.018028 | 0.010392 | 9.00E−02 | |
| | ASA PS | 0.029669 | 0.005382 | 1.86E−06 | * |
| | Male | −0.028225 | 0.009967 | 7.02E−03 | * |
| | Age | 0.021573 | 0.004606 | 2.83E−05 | * |
| THETA | Emergent | 0.000928 | 0.001383 | 5.06E−01 | |
| | ASA PS | 0.002965 | 0.000716 | 1.59E−04 | * |
| | Male | 0.002574 | 0.001327 | 5.89E−02 | |
| | Age | 0.003939 | 0.000613 | 8.74E−08 | * |

ASA PS—American Society of Anesthesiologists physical status,
SE—standard error

### Discussion

‘Low’ pressures, with mean arterial pressures (MAPs) typically below 65 mmHg have been linked to poor outcomes after non-cardiac surgery^{1,8}. Maintaining pressure just above these thresholds, however, may not minimize IOH risk as recent evidence indicates these pressures are strong predictors of impending IOH¹⁰⁻¹³. Thus, identifying pressures that possess a low risk of subsequent IOH are attractive as hemodynamic targets for patients undergoing surgery.

Provided herein is a high-performance preoperative prediction model to distinguish intraoperative MAP according to their risk of IOH and identify low-risk pressures before surgery. MAP targets were identified in 36 patient subgroups, each characterized by a unique combination of IOH risk factors. The major findings are 1) MAP associated IOH risk exhibits an exponential decay relationship where the rate of risk change increases as values approach 65 mmHg, 2) MAP targets for reducing risk of IOH range from 69 to 90 mmHg depending on subgroup and applied risk benchmark, 3) risk curve segments (plateau, exponential) and their transition are defined by two parameters, beta and theta, that are individually predicted by different patient factors.

These findings have several important implications. First, the model differentiates a wide range of clinically acceptable blood pressures according to individual patient factors prior to surgery. In young, male patients assigned a lower ASA physical status, IOH risk remains low until MAP nears 65 mmHg, at which point it rapidly increases. In patients with higher risks, the exponential increase begins earlier, at a higher MAP and from a higher plateau, suggesting these patients require greater clinical vigilance to anticipate/treat IOH events even when MAPs are well above 65 mmHg. This is particularly notable considering patients with elevated preoperative risks appear to be the most susceptible to IOH-induced end organ injury and those likely to benefit most from strategies that reduce exposure¹⁶.

Declining autonomic nervous system (ANS) function may contribute to greater MAP associated IOH risk, helping explain observed differences between subgroups. Women exhibit less sympathetic tone then men^{17,18}, and heart rate variability, an established surrogate for autonomic function, declines with increasing age¹⁹ and disease burden^{20,21}. Emergency surgery was not identified as an independent predictor of model parameters. This may be explained by the fact that it is more indicative of clinical decision-making than patient physiology as all types of patients may undergo emergent procedures.

Second, small differences in blood pressure translate to large differences in IOH risk, particularly at MAP nearest 65 mmHg. A MAPs of 70 mmHg was associated with nearly a 4-fold greater risk, on average across all subgroups studied, than 80 mmHg (16.1 vs 4.6%, data not shown), despite both being within acceptable limits recommended by current perioperative guidelines⁹.

The results reported in this example offer several strengths. Both training and validation datasets were sufficiently large to allow generalizability of the findings and assess model performance across many distinct patient types. Multiple models (including both linear and non-linear) were evaluated to identify a high-quality model with excellent fitness. The average SE per MAP value was less than 1×10E4 for all subgroups even in validation testing. By distinguishing IOH risk of multiple blood pressures, up to ten minutes following MAP measurement, the model predicts IOH exposure defined by more than single depressions below 65 mmHg, a feature with clinical impact as IOH-associated complications are dose-dependent.

In summary, MAP levels commonly seen during noncardiac surgery have uniquely associated IOH risks. Risk are predictable across widely different patient groups and can be harnessed to generate patient-specific, hemodynamic interventions that reduce IOH exposure and its ill-effects, while minimizing interventional efforts that also have associated risk.

MAP targets identified using the implementations described herein offer clinicians the unqualified advantage of identifying which MAPs, within a broadly acceptable range, minimize both risk for subsequent IOH and unnecessary pressure intervention, thus providing valuable information for hemodynamic management.

### REFERENCES

1. Wesselink E M, Kappen T H, Torn H M, Slooter A J C, van Klei W A. Intraoperative hypotension and the risk of postoperative adverse outcomes: a systematic review. Br J Anaesth. 2018; 121(4):706-721.
2. Saugel B, Sessler D I. Perioperative Blood Pressure Management. Anesthesiology. 2021; 134(2):250-261.
3. Bijker J B, van Klei W A, Vergouwe Y, et al. Intraoperative hypotension and 1-year mortality after noncardiac surgery. Anesthesiology. 2009; 111(6):1217-1226.
4. Monk T G, Bronsert M R, Henderson W G, et al. Association between Intraoperative Hypotension and Hypertension and 30-day Postoperative Mortality in Noncardiac Surgery. Anesthesiology. 2015; 123(2):307-319.
5. Salmasi V, Maheshwari K, Yang D, et al. Relationship between Intraoperative Hypotension, Defined by Either Reduction from Baseline or Absolute Thresholds, and Acute Kidney and Myocardial Injury after Noncardiac Surgery: A Retrospective Cohort Analysis. Anesthesiology. 2017; 126(1):47-65.
6. Sun L Y, Wijeysundera D N, Tait G A, Beattie W S. Association of intraoperative hypotension with acute kidney injury after elective noncardiac surgery. Anesthesiology. 2015; 123(3):515-523.
7. Walsh M, Devereaux P J, Garg A X, et al. Relationship between intraoperative mean arterial pressure and clinical outcomes after noncardiac surgery: toward an empirical definition of hypotension.

### Anesthesiology. 2013; 119(3):507-515.

8. Vernooij L M, van Klei W A, Machina M, Pasma W, Beattie W S, Peelen L M. Different methods of modelling intraoperative hypotension and their association with postoperative complications in patients undergoing non-cardiac surgery. Br J Anaesth. 2018; 120(5):1080-1089.
9. Sessler D I, Bloomstone J A, Aronson S, et al. Perioperative Quality Initiative consensus statement on intraoperative blood pressure, risk and outcomes for elective surgery. Br J Anaesth. 2019; 122(5):563-574.
10. Davies S J, Vistisen S T, Jian Z, Hatib F, Scheeren T W L. Ability of an Arterial Waveform Analysis-Derived Hypotension Prediction Index to Predict Future Hypotensive Events in Surgical Patients.

### Anesth Analg. 2020; 130(2):352-359.

11. Hatib F, Jian Z, Buddi S, et al. Machine-learning Algorithm to Predict Hypotension Based on High-fidelity Arterial Pressure Waveform Analysis. Anesthesiology. 2018; 129(4):663-674.
12. Kendale S, Kulkarni P, Rosenberg A D, Wang J. Supervised Machine-learning Predictive Analytics for Prediction of Postinduction Hypotension. Anesthesiology. 2018; 129(4):675-688.
13. Sudfeld S, Brechnitz S, Wagner J Y, et al. Post-induction hypotension and early intraoperative hypotension associated with general anaesthesia. Br J Anaesth. 2017; 119(1):57-64.
14. Collins G S, Reitsma J B, Altman D G, Moons K G, Group T. Transparent reporting of a multivariable prediction model for individual prognosis or diagnosis (TRIPOD): the TRIPOD statement. The TRIPOD Group. Circulation. Jan. 13 2015; 131(2):211-9.
15. R Core Team (2019). R: A language and environment for statistical computing. R Foundation for Statistical Computing V, Austria. URL https://www.R-project.org/.
16. Mathis M R, Naik B I, Freundlich R E, et al. Preoperative Risk and the Association between Hypotension and Postoperative Acute Kidney Injury. Anesthesiology. 2020; 132(3):461-475.
17. Koenig J, Thayer J F. Sex differences in healthy human heart rate variability: A meta-analysis. Neurosci Biobehav Rev. 2016; 64:288-310.
18. Shaffer F, Ginsberg J P. An Overview of Heart Rate Variability Metrics and Norms. Front Public Health. 2017; 5:258.
19. Bonnemeier H, Richardt G, Potratz J, et al. Circadian profile of cardiac autonomic nervous modulation in healthy subjects: differing effects of aging and biological sex on heart rate variability. J Cardiovasc Electrophysiol. 2003; 14(8):791-799.
20. Aeschbacher S, Schoen T, Dorig L, et al. Heart rate, heart rate variability and inflammatory biomarkers among young and healthy adults. Ann Med. 2017; 49(1):32-41.
21. Zhou X, Ma Z, Zhang L, et al. Heart rate variability in the prediction of survival in patients with cancer: A systematic review and meta-analysis. J Psychosom Res. 2016; 89:20-25.
22. Futier E, Lefrant J Y, Guinot P G, et al. Effect of Individualized vs Standard Blood Pressure Management Strategies on Postoperative Organ Dysfunction Among High-Risk Patients Undergoing Major Surgery: A Randomized Clinical Trial. JAMA. 2017; 318(14):1346-1357.
23. Hasanin A M, Amin S M, Agiza N A, et al. Norepinephrine Infusion for Preventing Postspinal Anesthesia Hypotension during Cesarean Delivery: A Randomized Dose-finding Trial. Anesthesiology. 2019; 130(1):55-62.
24. Brady K M, Hudson A, Hood R, DeCaria B, Lewis C, Hogue C W. Personalizing the Definition of Hypotension to Protect the Brain. Anesthesiology. 2020; 132(1):170-179.
25. Saugel B, Reese P C, Sessler D I, et al. Automated Ambulatory Blood Pressure Measurements and Intraoperative Hypotension in Patients Having Noncardiac Surgery with General Anesthesia: A Prospective Observational Study. Anesthesiology. 2019; 131(1):74-83.
26. Wijnberge M, Geerts B F, Hol L, et al. Effect of a Machine Learning-Derived Early Warning System for Intraoperative Hypotension vs Standard Care on Depth and Duration of Intraoperative Hypotension During Elective Noncardiac Surgery: The HYPE Randomized Clinical Trial. JAMA. 2020; 323(11):1052-1060.

In view of the many possible implementations to which the principles of the disclosed subject matter may be applied, it should be recognized that the illustrated implementations are only examples and should not be taken as limiting the scope of the description provided herein.

1. A method of generating a target mean arterial pressure (MAP) for a patient during a surgery, comprising:
calculating the target MAP based on the age, biological sex, emergent surgery status, American Society of Anesthesiology (ASA) physical status (PS) class of the patient, and a predetermined intraoperative hypotension (IOH) risk benchmark for the surgery; and wherein:
the target MAP is a MAP falling within the range of from 66 to 100 mmHg; and
treating the patient to maintain a MAP at or above the target MAP during the surgery reduces a risk of IOH during the surgery, a risk of administering an unnecessary therapy to avoid IOH during the surgery, and a risk of not administering a therapeutic MAP-decreasing action during the surgery.

2. The method of claim 1, wherein the IOH comprises a MAP of 65 mmHg or less.

3. The method of claim 1, wherein the IOH comprises a systolic blood pressure of 100 mmHg or less, and/or a decrease in blood pressure of at least 20% during surgery compared to preoperative blood pressure in the patient.

4. The method of claim 1, wherein the target MAP is a MAP falling within the range of from 65 to 100 mmHg.

5. The method of claim 1, wherein the target MAP is a MAP falling within the range of from 69 to 90 mmHg.

6. The method of claim 1, wherein the surgery is a non-cardiac surgery under general anesthesia.

7. The method of claim 1, wherein the surgery comprises MAP-modulating action comprising a therapy that increases or decreases MAP in the patient.

8. The method of claim 1, wherein treating the patient to maintain the MAP of the patient at or above the target MAP during the surgery comprises administering or increasing a MAP-increasing therapy and/or reducing a MAP-decreasing therapy in an amount effective to maintain the MAP of the patient at or above the target MAP.

9. The method of claim 1, wherein the predetermined IOH risk benchmark is an IOH risk of from 1% to 15% for historical patients with comparable surgery and age, biological sex, emergent surgery status, and ASA PS class as the patient for the current surgery.

10. The method of claim 1, wherein the predetermined IOH risk benchmark is an IOH risk of 5% or less for historical patients with comparable surgery and age, biological sex, emergent surgery status, and ASA PS class as the patient for the current surgery.

11. The method of claim 1, wherein the predetermined IOH risk benchmark is calculated as the percent of MAP readings between 65-100 mmHg followed by at least 1 MAP <65 mmHg within 10 minutes of their measurement for historical patients with comparable surgery and age, biological sex, emergent surgery status, and ASA PS class as the patient for the current surgery

12. The method of claim 1, further comprising conducting the surgery on the patient and treating the patient to maintain the MAP of the patient at or above the target MAP during the surgery.

13. The method of claim 1, wherein maintaining the MAP of the patient at or above the target MAP reduces the IOH risk for the patient compared to an average IOH risk for historical patients with comparable surgery and age, biological sex, emergent surgery status, and ASA PS class as the patient.

14. The method of claim 13, wherein the historical patients are prior patients from the same hospital system or surgical center as the patient.

15. The method of claim 1, wherein:
treating the patient to maintain the MAP of the patient at or above the target MAP reduces the risk of the IOH during the surgery by at least 50% compared to a control;
treating the patient to maintain the MAP of the patient at or above the target MAP reduces the risk of administering an unnecessary therapy to avoid IOH during the surgery by at least 50% compared to a control;
treating the patient to maintain the MAP of the patient at or above the target MAP reduces the risk of not administering a therapeutic MAP-decreasing action during the surgery by at least 50% compared to a control; and/or
treating the patient to maintain the MAP at or above the target MAP reduces a risk of surgical complication compared to a control.

16. The method of claim 15, wherein the surgical complication is acute kidney injury, stroke, intraoperative awareness, cerebral vascular accident, transient ischemic attack, postoperative cognitive dysfunction, myocardial injury or infarction, need for interventional revascularization, hospitalization due to heart failure within 30 days or 1 year following surgery, or death within 30 days or 1 year following surgery.

17. The method of claim 15, wherein the control is an average IOH risk, an average risk of administering an unnecessary therapy to the patient to avoid IOH, an average risk of not administering a therapeutic MAP-decreasing action, and/or an average risk of surgical complication, for historical patients with comparable surgery and age, biological sex, emergent surgery status, and ASA PS class as the patient.

18. The method of claim 17, wherein the historical patients are prior patients from the same hospital system or surgical center as the patient.

19. The method of claim 1, wherein the target MAP is calculated according to the patient status and target MAP ranges in following table:

| Patient Status | | | | |
|---|---|---|---|---|
| Emergent | ASA PS | Biological | | Target MAP |
| surgery? | Class | sex | Age | (mmHg) |
| No | 1 | F | 18-42 | 72-75 |
| No | 1 | M | 18-42 | 70-73 |
| No | 2 | F | 18-42 | 72-75 |
| No | 2 | F | 43-56 | 73-78 |
| No | 2 | F | 57-66 | 75-81 |
| No | 2 | F | >66 | 75-82 |
| No | 2 | M | 18-42 | 71-74 |
| No | 2 | M | 43-56 | 72-76 |
| No | 2 | M | 57-66 | 73-78 |
| No | 2 | M | >66 | 74-79 |
| No | 3 | F | 18-42 | 72-76 |
| No | 3 | F | 43-56 | 73-78 |
| No | 3 | F | 57-66 | 75-81 |
| No | 3 | F | >66 | 75-83 |
| No | 3 | M | 18-42 | 72-76 |
| No | 3 | M | 43-56 | 73-78 |
| No | 3 | M | 57-66 | 74-80 |
| No | 3 | M | >66 | 75-82 |
| No | 4 | F | 18-42 | 73-78 |
| No | 4 | F | 43-56 | 74-80 |
| No | 4 | F | 57-66 | 75-83 |
| No | 4 | F | >66 | 77-86 |
| No | 4 | M | 18-42 | 73-78 |
| No | 4 | M | 43-56 | 73-79 |
| No | 4 | M | 57-66 | 75-82 |
| No | 4 | M | >66 | 76-85 |
| Yes | 2 | M | 18-42 | 71-74 |
| Yes | 3 | F | >66 | 76-85 |
| Yes | 3 | M | 18-42 | 73-78 |
| Yes | 3 | M | 43-56 | 73-80 |
| Yes | 3 | M | 57-66 | 75-82 |
| Yes | 3 | M | >66 | 76-84 |
| Yes | 4 | M | 18-42 | 75-84 |
| Yes | 4 | M | 43-56 | 75-81 |
| Yes | 4 | M | 57-66 | 76-86 |
| Yes | 4 | M | >66 | 77-90 |

20. The method of claim 1, wherein the target MAP is calculated according to the patient status and target MAP ranges in following table:

| Patient Status | | | | |
|---|---|---|---|---|
| Emergent | ASA PS | Biological | | Target MAP |
| surgery? | Class | sex | Age | (mmHg) |
| No | 1 | F | 18-42 | 75 |
| No | 1 | M | 18-42 | 73 |
| No | 2 | F | 18-42 | 75 |
| No | 2 | F | 43-56 | 78 |
| No | 2 | F | 57-66 | 81 |
| No | 2 | F | >66 | 82 |
| No | 2 | M | 18-42 | 74 |
| No | 2 | M | 43-56 | 76 |
| No | 2 | M | 57-66 | 78 |
| No | 2 | M | >66 | 79 |
| No | 3 | F | 18-42 | 76 |
| No | 3 | F | 43-56 | 78 |
| No | 3 | F | 57-66 | 81 |
| No | 3 | F | >66 | 83 |
| No | 3 | M | 18-42 | 76 |
| No | 3 | M | 43-56 | 78 |
| No | 3 | M | 57-66 | 80 |
| No | 3 | M | >66 | 82 |
| No | 4 | F | 18-42 | 78 |
| No | 4 | F | 43-56 | 80 |
| No | 4 | F | 57-66 | 83 |
| No | 4 | F | >66 | 86 |
| No | 4 | M | 18-42 | 78 |
| No | 4 | M | 43-56 | 79 |
| No | 4 | M | 57-66 | 82 |
| No | 4 | M | >66 | 85 |
| Yes | 2 | M | 18-42 | 74 |
| Yes | 3 | F | >66 | 85 |
| Yes | 3 | M | 18-42 | 78 |
| Yes | 3 | M | 43-56 | 80 |
| Yes | 3 | M | 57-66 | 82 |
| Yes | 3 | M | >66 | 84 |
| Yes | 4 | M | 18-42 | 84 |
| Yes | 4 | M | 43-56 | 81 |
| Yes | 4 | M | 57-66 | 86 |
| Yes | 4 | M | >66 | 90 |

21. The method of claim 1, wherein the surgery comprises ear-nose-throat surgery, trauma surgery, urological surgery, neurosurgery, orthopedic surgery, vascular surgery, thoracic surgery, pediatric surgery, cardiac surgery, OB-GYN surgery, ophthalmologic surgery, transplant surgery, general surgery, plastic surgery, colon and rectal surgery, gynecologic oncology surgery, oral and maxillofacial surgery, critical care procedures comprising inhalation anesthetics, and dental surgical services or dental surgical services.

22. The method of claim 1, wherein treating the patient to maintain a MAP at or above the target MAP during the surgery comprises administering or modifying administration of fluids, a vasoactive agent, a vasodilator agent, an anesthetic agent, an opioid, a hypnotic, and/or an ancillary agent with known cardiovascular effects, to the patient.

23. The method of claim 1, wherein calculating the target MAP based on the age, biological sex, emergent surgery status, and ASA physical status class of the patient is performed at least in part using a computer.

24. A system for monitoring a patient during a surgery, comprising:
a hardware unit including a hardware processor and system memory;
a target mean arterial pressure (MAP) for the patient during the surgery stored in the system memory and calculated according to the method of claim 1;
a hemodynamic sensor coupled to the hardware unit;
a sensory alarm; and
wherein the hardware processor is configured to activate the sensory alarm if a MAP below the target MAP is measured for the patient during the surgery.

25. A computing system comprising:
at least one processor with memory attached thereto;
wherein the computing system is configured to execute instructions to perform a method according to claim 1.

26. One or more computer-readable media storing computer-readable instructions, which, when executed by one or more processors, cause a computer comprising the processors to perform the method of claim 1.
